(19) United States
(12) Patent Application Publication
(10) Pub. No.: US 2022/0280342 A1
(43) Pub. Date: Sep. 8, 2022

(54) INTUBATION SET FOR RECOVERY OF LIQUID DRAINAGE CAPABILITY OF THE TUBULAR ORGANS PROVIDING NASOLACRIMAL DRAINAGE

(71) Applicant: ONDOKUZ MAYIS UNIVERSITESI, Samsun (TR)

(72) Inventor: Volkan YETER, Samsun (TR)

(73) Assignee: ONDOKUZ MAYIS UNIVERSITESI, Samsun (TR)

(21) Appl. No.: 17/625,596

(22) PCT Filed: Jul. 9, 2020
(86) PCT No.: PCT/TR2020/050613

§ 371 Date: Jan. 7, 2022

(30) Foreign Application Priority Data
Jul. 9, 2019 (TR) 2019/10229

(51) Int. Cl. A61F 9/007

(52) U.S. Cl. CPC A61F 9/00772; A61B 17/3468

(57) ABSTRACT

It relates to an intubation set which enables treatment of organ damage, stenosis or obstruction such as punctal, canalicular and nasolacrimal duct etc. that provide drainage of liquid in the human body and provides recovery of the liquid drainage ability of these organs in an easy, fast and less injuring and less invasive manner.

### FIELD OF THE INVENTION

The invention relates to the recovery of liquid drainage capability of the tubular organs providing liquid drainage in the human body after they cannot function properly and/or damaged due to acquired or idiopathic reasons such as congenital reasons, inflammation, any kind of disease, drugs, chemicals, trauma etc.

The invention is particularly related to an intubation set which enables treatment of organ damage, stenosis or obstruction such as punctual, canalicular and nasolacrimal duct etc. that provide drainage of liquid in the human body and provides recovery of the liquid drainage ability of these organs in an easy, fast and less injuring and less invasive manner.

### STATE OF THE ART

After the tear produced by the lacrimal glands washes and moisturizes the eye surface, it is discharged into the nasal cavity by means of the lacrimal drainage system. The small openings on the upper and lower lids on the side of the eyelid closer to the nose are named as the upper punctum and lower punctum. The tear entering the upper and lower punctums with the movement of the eyelid reaches to the common canaliculus and then to the lacrimal sac with the help of the upper and lower canaliculus. It passes through the bone canal with nasolacrimal duct through the lacrimal sac; then it flows into an area which is between the inferior nasal conchae in the nose, intranasal base and intranasal lateral wall and is called inferior meatus, though valved opening. The tear accumulates in the patient's eye and thus the vision quality is distorted and at the same time the patient continuously requires to wipe his/her eyes who continuously shed tears since the tear cannot be discharged into the nose after stenosis or obstruction in this passage due to acquired or idiopathic reasons such as congenital reasons, inflammation, any disease, drugs, chemicals, trauma etc. Moreover, these symptoms decrease the quality of life of the patient.

Nasolacrimal duct is occluded in 2-6% of newborns. The most common reason of occlusion of nasolacrimal duct which can happen due to many acquired causes is idiopathic. In this case, the tear that cannot be discharged in the nasal cavity accumulates within the sac and can cause bacterial growth based on statis. Hypopyon and painful apsis of the incision site together with watering of the eyes can be seen in some patients.

The great majority of the congenital occlusions can be treated with lacrimal sac massage and antibiotic drops spontaneously up to one year age. In order to create the passage again in children who are not healed, probing and tube implantation can be recommended. The probing method is a method used for opening the occlusions that may happen along the nasolacrimal discharge paths by means of using metal probes with different sizes. If this method is not successful, silicone tube implantation can be recommendable.

As a result of the preliminary research made about the state of the art, patent file No U.S. Pat. No. 4,380,239 was analyzed. The invention subject to the application is a silicone tube implantation tool. There is a silicone tube with lumen of about 25-30 cm or without lumen between two metal probes having an olive-formed end with approximately 11 cm length and 0.4-0.5 mm diameter. The metal probe with olive-tip on one end of this tube system is inserted into one of the punctums, is passed through canaliculus, sac and nasolacrimal duct respectively and reaches to the inferior meatus. The olive-tip is caught from the intranasal section with its specific hook or is grasped by means of a holder by being monitored with the help of endoscopy and is removed from the nostril by being pulled. The silicone tube engaged with the metal probe advances along the whole channel with the metal probe and exits from the nostril. The metal probe with olive-tip on the other end of the silicone tube is removed from the nose in the same manner by means of inserting the same through the punctum and passing the same through the canaliculus, sac and nasolacrimal duct (namely by the silicone tube within the channel). Metal probes are removed by cutting. The two tips of the silicon tube which is removed from the nose are fixed to each other, are released into the nose or stitched to the side wall of the nose. When this tool and implantation method is applied; the surgeon can experience many difficulties. First, it is very difficult to view or monitor the area where the nasolacrimal duct is opened to the nose during surgery or examination. This makes the manipulation to be applied for catching or hooking the metal probe difficult in this field. The hard or flexible endoscopes may not always pass between the lateral intranasal wall and inferior nasal conchae during monitoring and sometimes the ecarteur and the concha may have to be elevated. The surgeons who do not have the opportunity of monitoring try to catch the metal probe with the tools that they insert into the nose in a manner such that they try to hit a target blindly. This requires some experience and capability. Sometimes this contact cannot be realized and the metal probe is removed and is inserted again in order to make sure that it is within the channel. The catching process inside the nose is repeated. This process without monitoring lasts within approximately 27 minutes and during these processes many traumas may be exerted on the nasolacrimal drainage system. This can lead to bond and occlusions again.

In the prior art, US2009281621A1 patent application devices used for normalizing the flow of fluid in tubular organs of human bodies that have been injured by a disease or an accident are disclosed. More specifically, the invention discloses to treating punctal, canalicular and nasolacrimal duct damage, stenosis or obstruction. In US2009281621A1, device contains an opening at the lower end of the guide sleeve. This guide sleeve is pushed into the nose from one of the puncta by passing a metal probe through its centre. A dyed liquid is delivered from the upper end lumen. When the dye is seen through the nostril, it is thought that the guide sleeve has passed to the appropriate place, the inferior meatus, and the metal probe in its lumen is removed. With the device disclosed in US2009281621A1, when the surgeon sees the dye in the nose, it is predicted that the stent has passed into the nose and is in the appropriate place. Therefore, the usage of this device has a disadvantage such as the necessity of using a dye to be sure about the location and necessity of internasal imaging.

As a result of the preliminary research made about the state of the art, intubation tool named MINI-MONOKA was analyzed. This tool consists of a single canaliculus passage short silicon tube which enables the tube to catch the punctum with plug fixation in the punctum. Since this tool is very short to reach the nasolacrimal duct, it can only be used for the canaliculus intubation; it does not provide a solution for the damage, stenosis or obstruction of the nasolacrimal duct (7).

In the state of the art, the document of the intubation tool commercially named as MASTERKA was analyzed. This tool is produced in 3 different models with 30 mm, 35 mmm and 40 mm lengths and on one end of the same, a metal tube is passed through the lumen of the system having the punctum fixing plug system. This metal is inserted through the punctum together with the silicon tube cover thereon, passes through the canalicular sac and nasolacrimal duct and is removed from the inferior nasal meatus. The silicone tube on the metal probe namely where the same passes through its lumen is kept at the level of the punctum and is removed from the metal probe lumen. Let us assume that the silicone tube is within the nasal cavity then it is fixed to the punctum by means of a plug. The drainage path of the patient is measured with the help of the measuring probe before this process and the appropriate model among these 3 models is determined and is implanted as it is described. Monitoring is required in this method used in canaliculus or punctum occlusions in order to understand whether the silicone tube reaches to the nasal cavity or not or it remains in this cavity or not. Moreover, the silicone tube has a form which can pass through a single canaliculus, it remains like an accordion by being accumulated on the upper portion of the narrowness in the presence of a very narrow nasolacrimal duct and even the metal probe can pass through the silicone tube by perforating the same. Moreover, while the metal probe in the lumen is removed, silicone tube returns from the punctum easily based on the friction between the silicone tube and the metal probe. Therefore its stability in the channel is reduced and can come out from the channel again.

As a result due to the abovementioned disadvantages and the insufficiency of the current solutions regarding the subject matter, a development is required to be made in the relevant technical field.

### BRIEF DESCRIPTION OF THE INVENTION AND THE AIM OF THE INVENTION

The invention is related to an intubation set which enables treatment of organ damage, stenosis or obstruction such as punctal, canalicular and nasolacrimal duct etc. that provide drainage of liquid in the human body and provides recovery of the liquid drainage ability of these organs in an easy, fast and less invasive manner.

The most important aim of the invention is to realize tube implantation without requiring monitoring the intranasal area and passing metal probe through the nose in case contact with the nose occurs.

Another important aim of the invention is to reduce the manipulation to be applied for catching or hooking the used metal probe and to enable the removal of the metal probe or the tube applied in the nose spontaneously.

Another aim of the invention is to realize the implantation within a shorter period of time. Another important aim of the invention is to provide an implantation which gives less damage to intranasal structures, is less traumatic, causes less mucosal bleeding and is less invasive by minimizing the intranasal intervention.

Another aim of the invention is to develop an intubation set which does not require experience during long years and ability and provides all surgeons to realize this intervention.

The most important advantage of the invention is to minimize the anesthesia process that the patient is subjected by reducing the implantation process.

The structural and characteristic features of the present invention will be understood clearly by the following drawings and the detailed description written with reference to these drawings.

Therefore, the evaluation shall be made by taking these figures and the detailed description into consideration.

### DESCRIPTION OF THE FIGURES

FIG. 1 is a drawing which gives the image of the nasolacrimal excretory organs, tubular organs.

FIG. 2 is a drawing which gives a side view of the bicanalicular tube implantation tool which can pass through both two punctums and canaliculus without metal probe in its lumen.

FIG. 3 is a drawing which gives the flattened view of the bicanalicular tube implantation tool which can pass through both two punctums and canaliculus after the metal probe is implanted on the front portion of the single guide tube lumen, by taking the form of the metal probe.

FIG. 4 is a drawing which shows the flattened form of the front portion of the guide tube of the nasolacrimal tube implantation tool which can pass through a single punctum and canaliculus (monocanaliculus) and with punctum plug-fixation hook after the metal probe is implanted.

FIG. 4A is a drawing which gives a side view of the punctum plug-fixation hook.

FIG. 4B is a drawing which gives a front view of the punctum plug-fixation hook.

FIG. 4C is a drawing which shows the steps of locating the puctum plug-fixation hook to the punctum.

FIG. 5 is a drawing which gives a side view of the flat shape of front portion of the guide tube when there is a metal probe in its lumen by entering through the front opening (16a) of the guide tube.

FIG. 5B is a drawing which shows the side view of the twisted shape of the front portion of the guide tube when the guide tube is pushed forward by removing the metal probe from the lumen of the front opening (16a) of the guide tube.

FIG. 5C is a drawing which shows the flattened middle and rear sections of the guide tube by entering through the rear opening (16b) of the guide tube and passing through the front opening of the metal probe in the lumen.

FIG. 6A is a drawing which shows the convex/parabolic/blunt guide tube tip which does not have magnetic property and ends by narrowing.

FIG. 6B Is a drawing which shows the guide tube tip which is narrowed and has metal and material with magnetic feature in its lumen.

FIG. 6C is a drawing which shows the convex/parabolic/blunt guide tube tip which has metal and material with magnetic feature in its lumen.

FIG. 6D is a drawing which shows the guide tube which has metal and material with magnetic feature created by entering into the guide tube.

FIG. 6E is a drawing which shows the guide tube tip which has liquid with magnetic feature in its lumen.

FIG. 6F is a drawing which shows the guide tube tip which has metal and material with magnetic feature on its wall.

FIG. 6G.1 is a drawing of the guide tube which has guide probe in its lumen to the endpoint and shows that the terminated tip is open.

FIG. 6G.2 is a drawing which shows the passage of the continuous or segmented magnetic material that is flexible, foldable and can be pushed from the other end through the guide tube lumen.

FIG. 6G.3 is a drawing which shows the passage of the continuous or segmented magnetic material that is flexible, foldable and can be pushed from the other end into the lumen through the guide tube lumen and the form after the it is removed from the guide tube tip.

FIG. 7 is the drawings which show the intranasal sagittal sections wherein the front portion of the guide tube is directed towards the nostril with the manipulation of the guide hook and shows the removal of the same with the medical tube from the nostril gradually in the nasolacrimal tube implantation technique realized with a normal tip guide tube.

FIG. 7A is a drawing which shows the intranasal sagittal section by directing the guide tube towards the nostril in the downward-front direction with the manipulation of the guide hook.

FIG. 7a1 is a drawing which shows the coronal section by directing the guide tube towards the nostril in the downward-front direction with the manipulation of the guide hook.

FIG. 7a2 is a drawing which shows the intranasal axial section by directing the guide tube towards the nostril in the downward-front direction with the manipulation of the guide hook.

FIG. 7B is a drawing which shows the intranasal sagittal section by directing the guide tube towards the nostril in the direction of nasal septum and upwardly with the manipulation of the guide hook.

FIG. 7b1 is a drawing which shows the coronal section by directing the guide tube towards the nostril in the direction of nasal septum and upwardly with the manipulation of the guide hook.

FIG. 7b2 is a drawing which shows the intranasal axial section by directing the guide tube towards the nostril in the direction of nasal septum and upwardly with the manipulation of the guide hook.

FIG. 7C is a drawing which shows the intranasal sagittal section by directing the guide tube towards the nostril in the upward-forward direction with the manipulation of the guide hook.

FIG. 7c1 is a drawing which shows the coronal section by directing the guide tube towards the nostril in the upward-forward direction with the manipulation of the guide hook.

FIG. 7c2 is a drawing which shows the intranasal axial section by directing the guide tube towards the nostril in the upward-forward direction with the manipulation of the guide hook.

FIG. 7D is a drawing which shows the intranasal sagittal section by directing the guide tube towards the nostril in the downward-forward direction with the manipulation of the guide hook.

FIG. 7d1 is the drawings which show the coronal section of the steps of directing the guide tube towards the nostril in the forward-horizontal direction with the manipulation of the guide hook.

FIG. 7d2 is the drawings which show the intranasal axial section of the steps of directing the guide tube towards the nostril in the forward-horizontal direction with the manipulation of the guide hook.

FIG. 8 is a drawing which shows the intranasal sagittal sections showing the guidance of the guide tube and medical tube out of the nostril gradually by means of catching the same with the magnet/electromagnet in the intranasal cavity, with the nasolacrimal tube implantation technique realized with a magnetic tip guide tube.

FIG. 9 is a drawing showing the intranasal sagittal sections of the guide tube which is misdirected backwardly in the nose due to different reasons, is caught by means of the specific hook in the nose and is removed out of the nose.

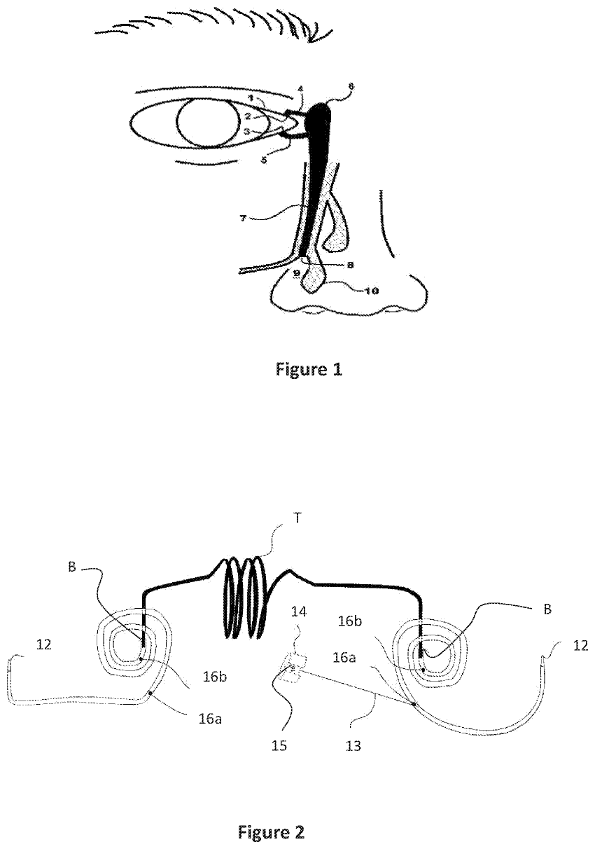

### REFERENCE NUMBERS

1. Upper eyelid
2. Upper eyelid punctum
3. Lower eyelid punctum
4. Upper canaliculus
5. Lower canaliculus
6. Tear sac
7. Nasolacrimal duct
8. Opening of nasolacrimal duct opened to the lower meatus
9. Inferior nasal meatus
11. Guide tube
12. Guide tube tip
13. Metal probe
14. Metal probe holder
15. Groove
16a. Front opening
16b. Rear opening
20. Forceps
21. material/magnet/electromagnet/holder with magnetic feature 22. Sickle tip rescue hook
23. Metal and material with magnetic feature
24. Magnetic fluid
25. Tube wall with magnetic feature
26. Extended structure with continuous and segmented magnetic feature that is flexible, foldable and can be pushed into the lumen.
L. Lumen
B. Connection part
Y. Guide clamp
V. Vestibule-mucosa border

### DESCRIPTION OF THE INVENTION

The invention is related to an intubation set which enables treatment of organ damage, stenosis or obstruction such as punctal, canalicular and nasolacrimal duct etc. that provide drainage of liquid in the human body and provide recovery of the liquid drainage ability of these organs in an easy, fast and less invasive manner. The subject of the invention is an intubation set which supports each of the monocanalicular and bicanalicular intubation.

On one hand, the bicanalicular intubation enables implantation by passing through both punctums and canaliculus; on the other hand the monocanalicular intubation enables implantation by passing through a single punctum and canaliculus.

The inventive tube and intubation set consists of the following,
metal probe (13),
metal probe holder (14) having a groove (15) that prevents the guide tube (11) from rotating by surrounding the same externally,
holder with various features for removing the guide tube (11) from the nostril by grasping the same; forceps (20), sickle tip rescue hook (22), material/magnet/electromagnet/holder with magnetic feature (21) in different geometric forms and structures,
guide clamp (Y) which prevents the guide tube (11) from rotating and changing its direction; enables to fix the guide tune while it is shifted towards the nasal cavity over the metal probe (13),
medical tube or medical intubation tube with punctum plug-fixation clamp used in the intubation of nasolacrimal drainage paths for the bicanalicular or monocanalicular intubation application,
guide tube (11) comprising a guide tube tip (12) with various structure and features on one end that provides inlet from punctums, an opening thereon through which the metal probe (13) passes and is directed in the nose and a connection part (B) on the other end where its connection with the medical tube is enabled, having various features, numbers and formed with various slopes, with magnetic feature or without the same.

The guide tube tip portion (12) which is flexible, folding and can be pushed into the lumen (L), has continuous or segmented magnetic feature, enables the passage of the extended structure (26) into the nose, is completely open or completely closed, comprises metal or material with magnetic feature (23) or magnetic fluid (24) or tube wall with magnetic feature (25).

Since there is an extended structure (26) at the end of the guide tube tip (2) which is flexible, foldable and can be pushed into the lumen (L), has continuous or segmented magnetic feature, it is provided to catch this structure by means of the material/magnet/electromagnet/holder with magnetic feature (21) by means of pushing the same outwardly and thus the intranasal invasion is minimized.

In a preferred embodiment of the invention; there is at least one opening through which the metal probe (13) passes and is directed into the nose, there may be two openings as front opening (16a) and/or rear opening (16b). Classification of the opening as front and back; in the presence of the guide tube (11) which is inserted through the front opening (16a) during the process and is misdirected; situations such as folding of the guide tube (11) in the nose or compressing of the same in a roll-like structure or misdirecting the same backwardly may occur. In this case; the metal probe (13) enters from the rear opening (16b) and the front opening (16a) is made flat by passing through the lumen. Afterwards, the rear section of the guide tube (11) with spiral structure is inserted into the nose manually or with the help of a holder and thus a guide tube (11) spiral accumulation is formed in that region by increasing the number and length of the rings formed in the nose. Therefore, the guide tube (11) can be caught easily in the nose by means of a specific sickle tip rescue hook (22) which is not traumatic.

The distance between the front opening (16a) and the rear opening (16b) is 5-30 cm, is typically between 8 cm-13 cm. The number of these openings can be increased based on demand.

Information about the functional and structural features of the set content separately is as the following.

Guide tube (11) comprising a guide tube tip (12) with various structure and features on one end that provides inlet from punctums, front and rear openings (16a) (16b) thereon to which the metal probe (13) passes through and is directed in the nose and a connection part (B) on the other end where its connection with the medical tube is enabled, having various features, numbers and formed with various slopes,
may consist of different polymer or monomers with semi-flexible structure and form taking feature, produced from poliamid, polyurethane, polyolefin, polysilicates, siloxanes, derivatives and mixtures thereof.
its external diameter can be between 0.1-4 mm, typically between 0.75-1 mm.
its lumen (L) diameter, internal diameter can be between 0.1-3.9 mm, typically between 0.20-0.85 mm.
it may be manufactured in various regular or irregular curvatures such as full round, crescent form, typically it is shown in crescent form.
its length is approximately between 5-30 cm, typically between 10-13 cm.
the distance between the guide tube tip (12) and the opening through which the metal probe (13) enters the guide tube lumen (L) is between 3 cm and 30 cm, typically between 8 cm and 15 cm.

In an embodiment where there is front and rear opening (16 a) and (16b); the distance between the guide tube tip (12) and the front opening (16a) through which the metal probe (13) enters the guide tube lumen (L) is between 3 cm and 30 cm, typically between 8 cm and 15 cm.

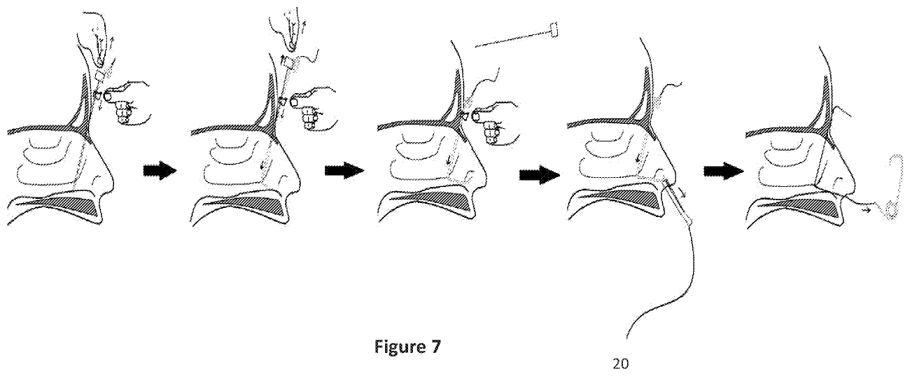

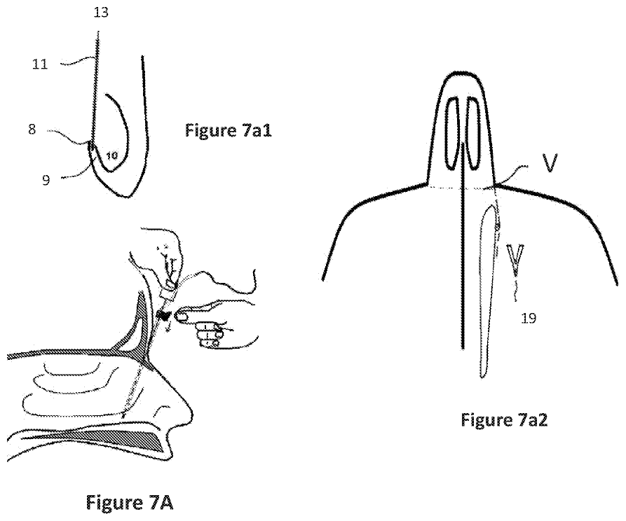

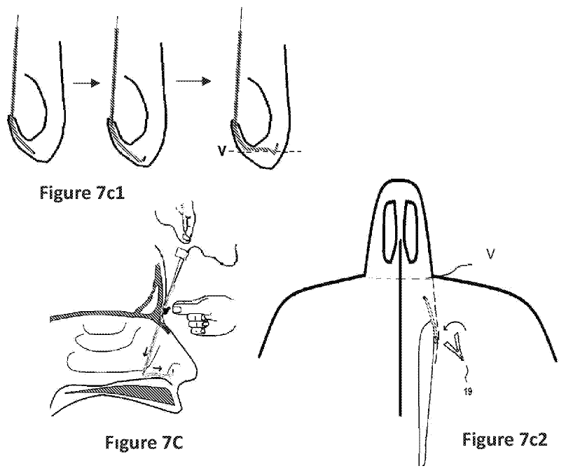

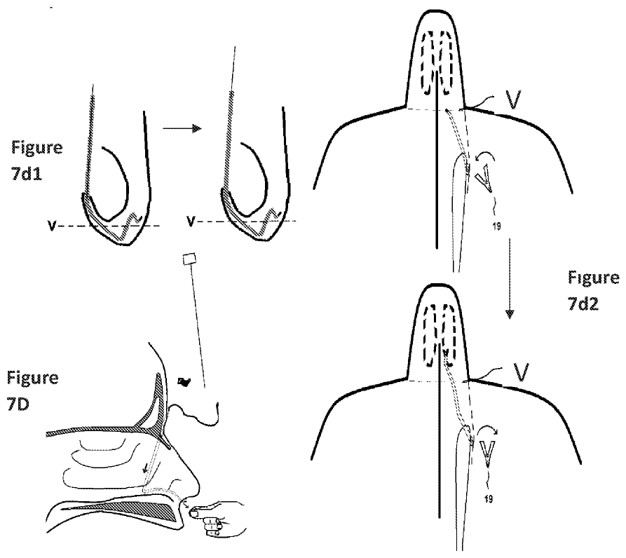

In order to make the surgeon to understand the level of the implanted tube, there are dimensional lines, markers and colors showing the bending direction of the tube thereon as shown in FIGS. 7A, 7B, 7C and 7D. thus, the surgeon is sure about which direction he/she inserts the tube or whether he/she guides the same in the nose in a forward direction or not.

Metal probe (13) which enters into the guide tube (11) and is directed in the nose,
may be manufactured from any material which is flexible, durable, semi-rigid, orientable, non-fragile, smooth reducing friction and with a feature that may give the guide tube (11) its own flat structure, also it may be manufactured from different materials with magnetic feature. If it is manufactured from magnetic materials, it will be facilitated to catch the same by means of magnet or electromagnet apparatuses in the nose. It is typically produced from steel.
may be with different geometric structure. Its diameter is between 0.1 mm and 3.9 mm, typically between 0.1 and 1.1 mm. Its length is between approximately 3-30 cm, typically between 10 cm and 15 cm. It is typically in the form of cylinder.

Metal probe holder (14) which is positioned on the distal end of the metal probe (13) having a groove (15) thereon that prevents its orientation to the side and rear directions after the guide tube (11) front section is lowered into the nasal cavity, keeps the upper portion of the tube in the same direction, has an adjustable position and form in relation with the structure of the guide tube (11), enables orientation of the metal probe (13),
has a feature which can be flattened or can take various forms for easy handling.

Guide clamp (Y) which is located in the central part of the guide tube (11), prevents the bottom half of the guide tube (11) from rotating in other directions and shows the surgeon the orientation direction of the tube while the guide tube (11) is scrapping towards the nose over the probe,
can be made with a design and different forms such that it does not give damage to the guide tube (11) while holding the same.
can be positioned in the required region or the guide tube (11) with the help of its attachable and detachable structure.

The medical tube which enables the nasolacrimal passage to be open during months in the drainage paths having guide tubes (11) on both ends formed with various slopes in the bicanalicular intubation process,
has bio-compatible feature.
has a feature which can easily take the form of the channel consisting of silicone, Teflon, polyamide and polymers similar to these and can shape the channel.
has a diameter between approximately 0.5-1.3 mm.
has a height varying between 15-35 cm.

There is a further punctum plug-fixation clamp which enables the medical tube to be fixed to the punctum in addition to the medical tube element in the content of the medical intubation tube with punctum plug-fixation for monocanalicular intubation. This clamp is located on the other end of the medical tube which is not engaged with the guide tube (11), in case it is fixed to the punctum, it enables the medical tube to remain fixed within the nasolacrimal channel.

The holder with various features in order to remove the guide tube (11) from the nostril by holding the same,

Holder types, forceps (20), sickle tip rescue hook (22), material/magnet/electromagnet/holder with magnetic feature (21) in different geometric forms and structures.

The overall diameter of the material/magnet/electromagnet/holder with magnetic feature (21) is between 1 mm and 2 cm—preferably between 0.1 cm and 0.5 cm.

This sickle tip rescue hook (22) can be manufactured with magnetic feature. The end of the sickle tip rescue hook (22) can be covered with a soft polymer like silicone.

The cavity extending inwardly from the nostrils to the interior sections, having a hairy skin surface is called as vestibule. When this area ends, the mucosa starts with the wet surface of the nose. The border of these two areas was named as vestibule-mucosa border (V). The nasal valve is this section where the nasal passage is the narrowest and the most convex section frontward of the intranasal base. When the tube passes from here, it will no more experience any obstacle in order to exit the nostril spontaneously. The inventive intubation set is developed such that it is able to pass this vestibule-mucosa border (V) without any damage or with minimum damage.

Guide tube tip (12) wherein,
its diameter has a structure which can be terminated by extending, remaining fixed, narrowing from the guide to the very end.
it may be in the curved/parabolic (blunt) form, olive-form, mushroom-form or open-ended cylinder form.
it may be produced from the same material as the guide tube (11) and also many be produced from a material with magnetic feature.
it may be produced from the material with magnetic feature such that it contains material with magnetic feature on the wall of the guide tube (11), metal or material with magnetic feature (23) in its lumen (K) and magnetic fluid (24) in its lumen (L).

The metal or material with magnetic feature (23) may be any magnetic material, particularly iron, nickel, cobalt, different metals and alloys with magnetic feature and/or magnets consisting of rare magnetic metals such as neodymium, samarium, cobalt etc.

The external diameter of the guide tube tips (12) with magnetic feature and various forms is between 0.1-3 mm, typically between 0.2-1.4 mm.

The internal lumen of the guide tube tip (12) with magnetic feature is between 0.1-2.9 mm, typically between 0.2-1.3 mm.

The height of the guide tube tip (12) with magnetic feature changes between 0.1 mm and 4 cm, typically between 0.1 mm and 1 cm.

Metal or materials with magnetic feature (23) can remain within the lumen (L) in an injected form and/or in a single form or in segmented form at the guide tube tip (12).

Guide tube tip (12) can be opened such that it can provide the passage of an extended structure with continuous and segmented magnetic feature that is flexible, foldable and can be pushed into the lumen (26).

The structural and functional information regarding the guide tube (11), metal probe (13) and the medical tube elements are indicated herein below.

The guide tube (11) is joined with the medical tube in the connection part (B). This combination can be obtained by the medical adhesives or by compressing and placing the medical tube in the lumen (L) of the guide tube (11) without using any adhesive.

The metal probe (13) enters the cavity (lumen) within the guide tube (11) through the opening where the metal probe enters the guide tube (11). These openings can be at least one opening and can be a front opening (16a) or rear opening (16b).

The metal probe (13) which enters the guide tube (11) through the front opening (16a) where the metal probe (13) enters the guide tube lumen (L) as shown in FIGS. 2,3,4, and 5 comprises a metal probe holder (14) which is positioned on the distal end and a groove (15) on this metal probe holder (14) which prevents its orientation to the side and rear directions after the guide tube (11) is lowered into the nasal cavity, keeps the upper portion of the tube in the same direction, has an adjustable position and form in relation with the structure of the guide tube (11). It is ensured that the guide tube (11) can be pushed and oriented in the nose in the forward-required direction by means of this groove (15). The location of the groove (15) can be changed and modified according to the shape of the tube.

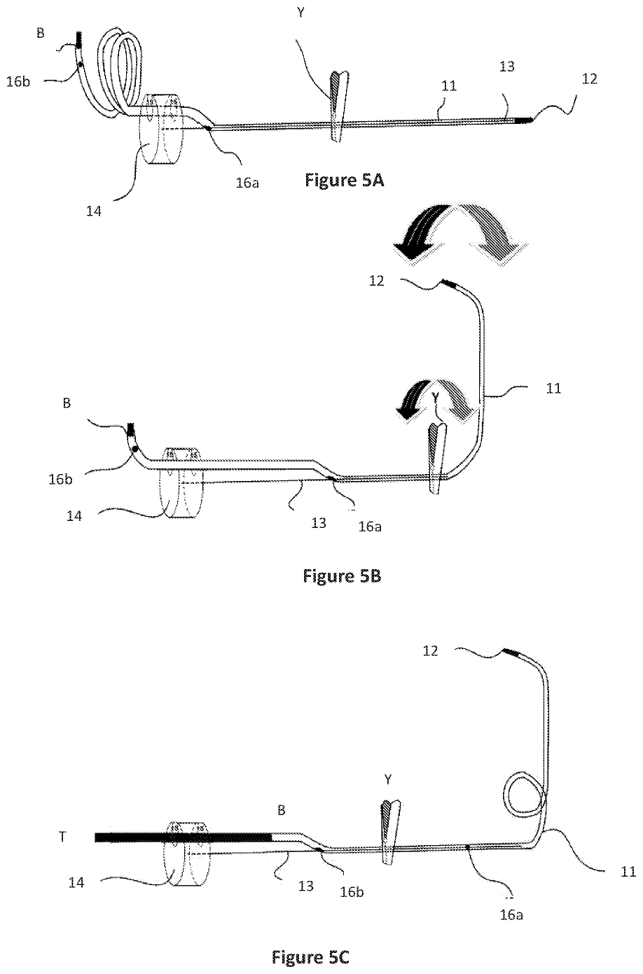

The flat form of the metal probe (13) gained when it is within the front section of the guide tube (11) is shown in FIG. 5A and bending of the half of the guide tube tip (12) when the probe is started to be removed is shown in FIG. 5B. In FIG. 5C, it is seen that the metal probe (13) passing through the rear opening (16b) flattens the curved rear portion and flattens the rear and middle portions of the guide tube (11) by passing through the front opening (16a).

The groove (15) in the metal probe holder (14) keeps the upper portion of the tube in the same direction in order to prevent the orientation of the guide tube (11) to the side and rear directions after it is lowered into the nasal cavity.

The guide clamp (Y) which is located in the central part of the guide tube (11), prevents the bottom half of the guide tube (11) from rotating in other directions and shows the surgeon the orientation direction of the tube while the guide tube (11) is scrapping towards the nose over the metal probe (13). This guide clamp (Y) can be made with a design and different forms such that it does not give damage to the guide tube (11) while holding the same and its location can be changed by attaching and detaching the guide tube (11) to the required section.

The structural and functional information of the guide tube tip (12) is detailed herein below.

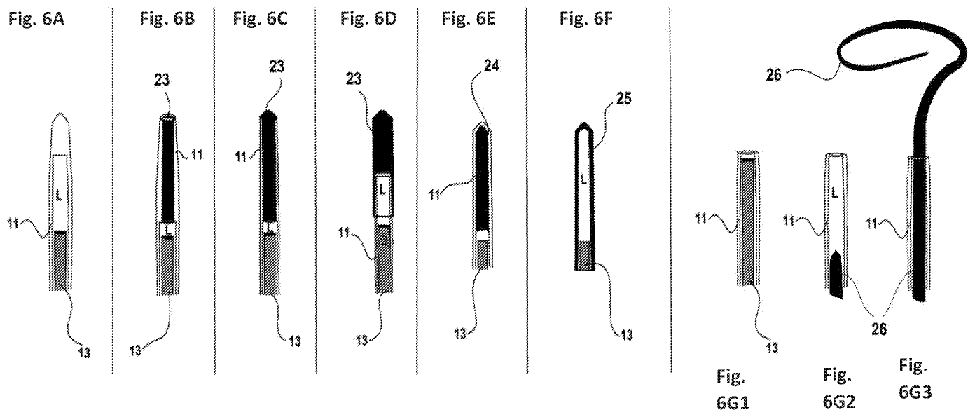

Some of the guide tube tips (12) with magnetic features and without magnetic features are summarized in FIG. 6. The guide tube tip (12) indicated in FIG. 6B is narrowed and terminated with an open end. In the lumen (L) extending to the open end, iron, nickel, cobalt with magnetic feature, different metals and alloys with magnetic feature and/or magnets consisting of rare magnetic metals such as neodymium, samarium, cobalt etc. or metal or materials with newly developed magnetic feature can be used. The metal or material with magnetic feature (23) located in the guide tube (11) may protrude beyond the lumen (L) as in FIG. 6C and may have various forms such as parabolic, convex, mushroom-form, olive-form etc. The metal or material with magnetic feature (23) compressed to the lumen (L) in FIGS. 6B and 6C can be tightly attached, compressed, adhered during the implantation so as not to fall into the channel from the lumen (L) with the repulse effect of the metal probe (13) or 1 mm or more space can be left between the metal probe (13) and metal or material with magnetic feature (23). Lumen-ends wherein the metal probe (13) may enter and the guide tube (11) may terminate, comprise metal or materials with magnetic feature (23) or magnet as shown in FIG. 6D. The external diameter of the guide tube tips (12) with various forms having metal or material with magnetic feature (23) is between 0.1 mm and 3 cm, typically between 0.2 mm and 1.4 cm. The internal lumen of the guide tube tip (12) with lumen having metal or material with magnetic feature (23) in FIG. 6 is between 0.1 mm and 2.9 cm, typically between 0.2 mm and 1.3 cm. The height of the guide tube tips (12) having metal or material with this magnetic feature (23) changes between 0.1 mm and 4 cm, typically between 0.1 mm and 1 cm. The guide tube tips (12) which have metal or material with magnetic feature (23) can either be in a single piece or in segments.

In FIG. 6E, the guide tube tip (12) with magnetic fluid (24) in its lumen (L) is shown. After the guide tube (11) having magnetic fluid (24) that circulates freely in the guide tube lumen (L) is forced into the nasal cavity and the metal probe (13) is removed and taken away from the lumen (L), it can be injected through the opening that the metal probe (13) enters the guide tube (11) or can be placed in the opening between the metal probe (13) and the guide tube lumen (L) before the implantation.

The guide tube tip (12) where the guide tube (11) wall is magnetic and the guide tube wall with magnetic feature (25) is shown in FIG. 6F. The tube wall with magnetic feature (25) is formed as a consequence of adding small size magnetic materials during the production of the guide tube (11) with different techniques and thus the guide tube (11) itself or only a section will have magnetic feature.

The guide tube (11) tip in FIG. 6G has an open end, after it is lowered in the nose with the help of metal probe (13)(FIG. 6G.1); the metal probe (13) is removed and an extended structure (26) which is flexible, foldable in the nose, can be pushed through the openings (16a and 16b) into the lumen (L), may have continuous or segmented magnetic feature can be lowered in the nose (FIG. 6G.2 and FIG. 6G.3).

The cavity extending inwardly from the nostrils to the interior sections, having a hairy skin surface is called as the nasal vestibule. When this area ends, the mucosa starts with the wet surface of the nose. The border of these two areas is named as vestibule-mucosa border (V). When the guide tube (11) passes the vestibule-mucosa border (V) where the nasal passage is the narrowest and the most convex section frontward of the intranasal base, that it exits the nostril spontaneously will not meet any obstacle. The inventive intubation set has been developed such that it is able to advance easily, quickly and at least invasively particularly from punctum to the vestibule-mucosa border (V) and from this border to the nasal outlet as shown in FIGS. 7a2, 7b2, 7c1, 7c2. Moreover, together with the guide tube (11), guide tube tip (12), medical tube, metal probe (13) features of which are indicated, the tube implantation without requiring monitoring the intranasal area and passing metal probe (13) through the nose in case contact with the nose occurs is provided.

The tube implantation methods of the inventive intubation set are described below.

When non-magnetic (normal) tube intubation method is applied, the function and details for usage of the inventive intubation set are given herein below.

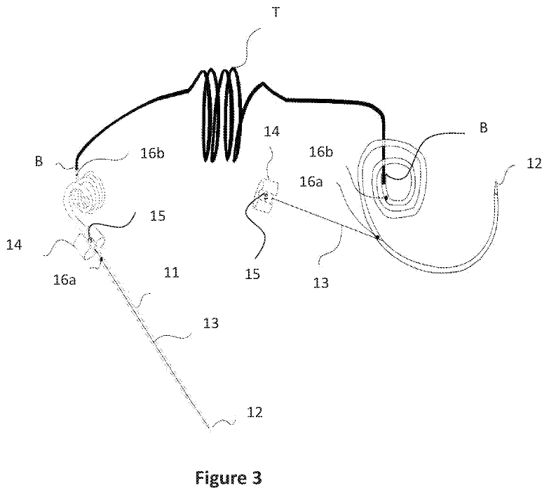

With the bicanaliculus implantation: the guide tube (11) is brought to its flat form in FIG. 3 from its form shown in FIG. 2 by means of passing the metal probe (13) through the guide tube lumen (L). The upper eyelid punctum (2) on the upper eyelid (1) and the lower eyelid punctum (3) on the lower eyelid where tube implantation will be realized are enlarged with the dilatator. It is ensured that there is a passage from punctums to the nasal cavity by using bowman probes. End of the guide tube (12) is pushed into the upper canaliculus (4) or lower canaliculus (5) and tear sac (6) respectively after passing through the lower eyelid punctum (3) or upper eyelid punctum (2) horizontally. When it is certain that the inner side wall of the tear sac (6) is reached, the tube is located upright by passing from horizontal plane to the vertical plane. The guide tube (11) which has a metal probe (13) in its lumen (L) is pushed in the nasolacrimal duct (7) after it is made upright as in the probing process and it is enabled to reach the inferior nasal meatus (9) by proceeding to the opening of nasolacrimal duct opened to the lower meatus (8). After this step, the guide tube (11) serves as a guide by holding the probe (13) in its lumen (L) in a stable manner without removing the same. Accordingly, as can be seen in FIG. 5A and FIG. 5B, the guide tube (11) can be rotated to the right or left easily in the direction required by the surgeon from the top such that there is a metal probe (13) in its center and it can rotate about the metal probe (13). Moreover, the metal probe (13) in the guide tube (11) remains within the guide tube (11) during the process until the end of the process and the guide tube (11) is pushed downwardly in a manual manner or by means of the guide clamp (Y) in the required direction. The process is continued with pushing the guide tube (11) to the nasolacrimal passage up to the level of the opening where the metal probe (13) enters the guide tube (11) until the probe (13) is totally removed.

It is ensured that the guide tube (11) is in the required direction. The guide tube (11) is seen in the nasal vestibule on the side where the intubation is realized and it is removed from the nostril by means of forceps (20) or a holder. The drawing which gives the intranasal sagittal sections showing the process steps gradually up to this phase is given in FIGS. 7A, 7B, 7C and 7D. The same process is repeated for the guide tube which provides passage through the other punctum. Two guide tubes (11) which are removed from the nostril are cut and taken away; the ends of the medical tube are shortened and knotted. The knot is released in the nose or it is fixed to the side wall of the nose vestibule and the process is ended.

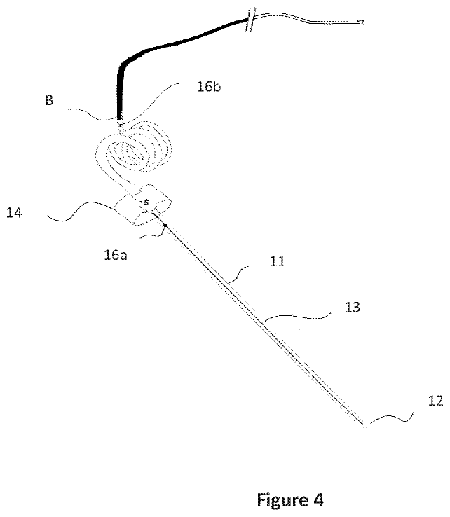

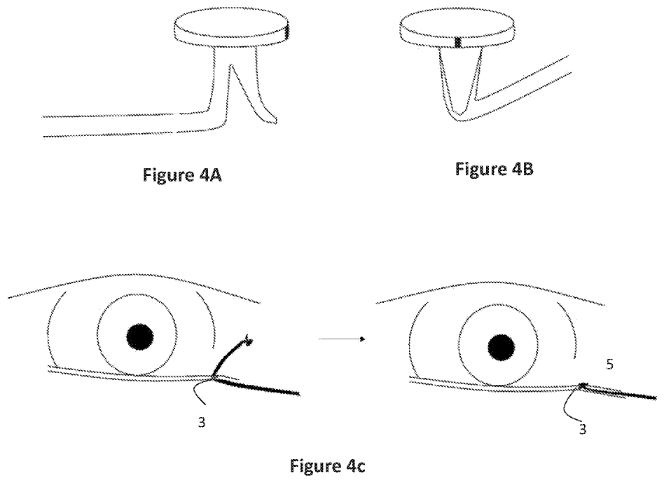

With the monocanaliculus implantation: The guide tube (11), as shown in FIG. 4, is made flat by passing the metal probe (13) through the guide tube lumen (L). The upper eyelid punctum (2) and the lower eyelid punctum (3) where tube implantation will be realized are enlarged with the dilatator. It is ensured that there is a passage from punctums to the nasal cavity by using bowman probes. the guide tube tip (12) is pushed into tear sac (6) by passing through the upper canaliculus (4) or lower canaliculus (5) respectively after passing through the lower eyelid punctum (3) or upper eyelid punctum (2) horizontally. When it is certain that the inner side wall of the tear sac (6) is reached, the guide tube (11) is located upright by passing from horizontal plane to the vertical plane. The guide tube (11) which has a metal probe (13) in its lumen (L) is pushed in the nasolacrimal duct (7) after it is made upright as in the probing process and it is enabled to reach the inferior nasal meatus (9) by proceeding to the opening of nasolacrimal duct opened to the lower meatus (8). As mentioned in the FIG. 5A and FIG. 5B, after this step, the guide tube (11) serves as a guide by holding the metal probe (13) in its lumen (L) in a stable manner without removing the same. Accordingly, the guide tube (11) can be rotated to the right or left easily in the direction required by the surgeon from the top such that there is a metal probe (13) in its center and it can rotate about the metal probe (13).

Moreover, the metal probe (13) in the guide tube (11) remains within the guide tube (11) during the process until the end of the process and the guide tube (11) is pushed downwardly in a manual manner or by means of the guide clamp (Y) in the required direction. The process is continued with pushing the guide tube (11) to the nasolacrimal passage up to the front opening (16a) where the metal probe (13) enters the guide tube (11) until the probe (13) is totally removed. The guide tube (11) is seen in the nasal vestibule where intubation is realized and it is removed from the nostril by means of forceps (20) or a holder. The drawing which gives the intranasal sagittal sections showing the process steps gradually up to this phase is given in FIG. 7. Then, the clamp punctum plug fixation is fixed to the punctum by means of pulling the guide tube (11) from the bottom. The guide tube (11) which is removed from the nostril is cut and taken away; the end of the medical tube is shortened and knotted. The knot is released in the nose or it is fixed to the side wall of the nose vestibule and the process is ended.

When the magnetic tube intubation method is used, the function and details for usage of the inventive intubation set are given herein below.

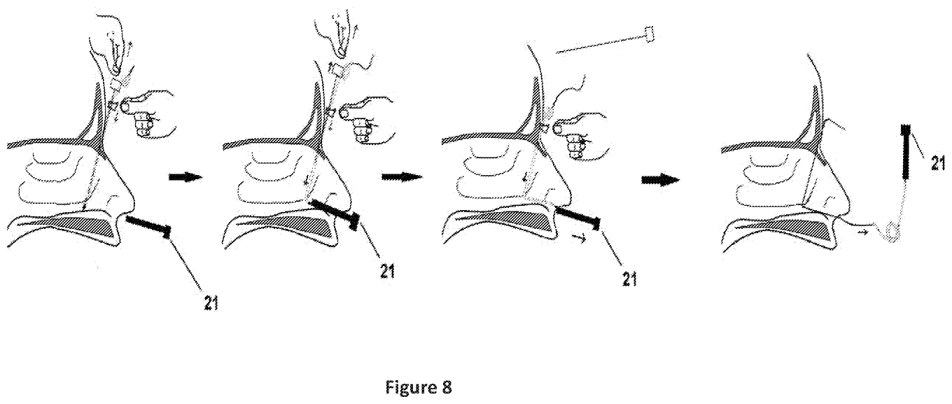

Bicanalicular implantation: The guide tube (11) having guide tube (11) with magnetic feature or guide tube tip (12) with magnetic feature is brought to its flat form in FIG. 3 by means of passing the metal probe (13) through the guide tube lumen (L) from its form shown in FIG. 2. The upper eyelid punctum (2) on the upper eyelid (1) and the lower eyelid punctum (3) on the lower eyelid where the tube implantation will be realized are enlarged with the dilatator. It is ensured that there is a passage from punctums to the nasal cavity by using bowman probes. the guide tube tip (12) is pushed into the upper canaliculus (4) or lower canaliculus (5) and tear sac (6) respectively after passing through the lower eyelid punctum (3) or upper eyelid punctum (2) horizontally. When it is certain that the inner side wall of the tear sac (6) is reached, the tube is located upright by passing from horizontal plane to the vertical plane. The guide tube (11) which has a metal probe (13) in its lumen (L) is pushed in the nasolacrimal duct (7) after it is made upright as in the probing process and it is enabled to reach the inferior nasal meatus (9) by proceeding to the opening of nasolacrimal duct opened to the lower meatus (8). A material/magnet/electromagnet/holder with an overall diameter between 1 mm and 2 cm-preferably between 0.1 cm and 0.5 cm—with magnetic feature, a collapsible clamp which has a magnetized end or alligator or holders such as forceps (20) or magnetized sickle-tip rescue hook (22) are directed from the nostril on the intubated side towards the base of the nose in a parallel manner and to the side wall of the nose, inferior nasal meatus (9). Thus, catching the guide tube tip (12) in the nose is facilitated. Following this phase, when it is ensured that the guide tube (11) is in the required direction and while the metal probe (13) is removed from the lumen (L), the guide tube (11) is pushed downwardly in a manual manner or by means of a guide clamp (Y). The process is continued until the guide tube (11) is pushed to the nasolacrimal passage up to the front opening (16a) where the metal probe (13) enters the guide tube (11) and until the metal probe (13) is totally removed and magnetic adhesion is provided with the guide tube tip (12) by providing its contact with the material/magnet/electromagnet/holder with magnetic feature (21) in the nostril. The guide tube (11) is removed from the nostril through the nasal vestibule on the side where intubation is realized. The drawing which gives the intranasal sagittal sections showing the process steps gradually up to this phase is given in FIG. 8. The same process is repeated by passing the guide tube (11) with magnetic feature on the other end of the vehicle shown in FIGS. 2 and 3 through the other punctum and canaliculus. Two guide tubes (11) which are removed from the nostril are cut and taken away; the ends of the medical tube are shortened and knotted. The knot is released in the nose or it is fixed to the side wall of the nose vestibule and the process is ended.

Monocanalicular implantation: The guide tube (11) having guide tube (11) wall with magnetic feature or guide tube tip (12) with magnetic feature is brought to its flat form in FIG. 3 by means of passing the metal probe (13) through the guide tube lumen (L) from its form shown in FIG. 2. The upper eyelid punctum (2) on the upper eyelid (1) and the lower eyelid punctum (3) on the lower eyelid where tube implantation will be realized are enlarged with the dilatator. It is ensured that there is a passage from punctums to the nasal cavity by using bowman probes. The guide tube tip (12) is pushed into the upper canaliculus (4) or lower canaliculus (5) and tear sac (6) respectively after passing through the lower eyelid punctum (3) or upper eyelid punctum (2) horizontally. When it is certain that the inner side wall of the tear sac (6) is reached, the tube is located upright by passing from horizontal plane to the vertical plane. The guide tube (11) which has a metal probe (13) in its lumen (L) is pushed in the nasolacrimal duct (7) after it is made upright as in the probing process and it is enabled to reach the inferior nasal meatus (9) by proceeding to the opening of nasolacrimal duct opened to the lower meatus (8). A material/magnet/electromagnet/holder with an overall diameter between 1 mm and 2 cm-preferably between 0.1 cm and 0.5 cm—with magnetic feature, a collapsible holder which has a magnetized end or magnetized sickle-tip rescue hook (22) are directed from the nostril on the intubated side towards the base of the nose in a parallel manner and to the side wall of the nose, inferior nasal meatus (9). Following this phase, when it is ensured that the guide tube (11) is in the required direction and while the metal probe (13) is removed from the lumen (L), the guide tube (11) is pushed downwardly in a manual manner or by means of a guide clamp (Y). The process is continued until the guide tube (11) is pushed to the nasolacrimal passage up to the front opening (16a) where the metal probe (13) enters the guide tube (11) and until the metal probe (13) is totally removed and magnetic adhesion is provided with the guide tube tip (12) by providing its contact with the material/magnet/electromagnet/holder with magnetic feature (21) in the nostril. The guide tube (11) is removed from the nostril through the nasal vestibule on the side where intubation is realized. The drawing which gives the intranasal sagittal sections showing the process steps gradually up to this phase is given in FIG. 8. The punctum plug fixation is fixed to the punctum by means of pulling the guide tube (11) from the bottom. The guide tube (11) which is removed from the nostril is cut and taken away; the end of the medical tube is shortened and knotted. The knot is released in the nose or it is fixed to the side wall of the nose vestibule and the process is ended.

While magnetic tube intubation technique is applied with the inventive intubation set, a magnetic field is created between the guide tube tip (12) and the holders since the guide tube tip (12) and holders with various features contain electromagnetic and/or different magnetic features. Therefore, the guide tube (11) may advance without requiring manual intensive physical intervention and without damaging the channels where it moves and thus the manipulation to be applied in the nose is reduced and the metal probe (13) or the tube applied in the nose is removed from the nose spontaneously.

When the tube intubation method is applied with the hook, the function and details for usage of the inventive intubation set are given herein below.

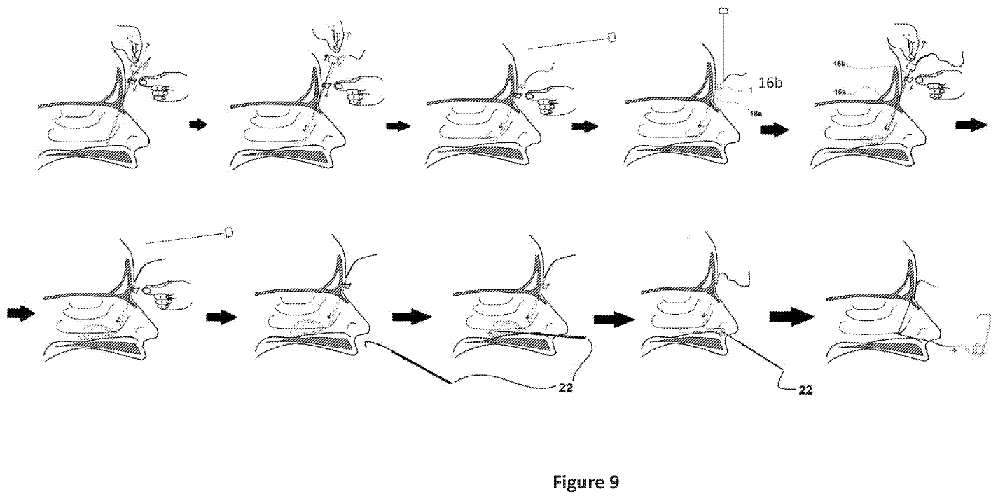

Intranasal anomalies or adhesions in some patients may lead to spontaneous removal of the guide tube (11) from the nostril or not contacting the tube tip to the magnetic holder. In the presence of the guide tube (11) in these patients which cannot be guided properly; the guide tube (11) is folded in the nose or can be compressed in a structure like a roll or can be misdirected backwardly. (In these cases; the metal probe (13) enters from the rear opening (16b) and the front opening (16a) is made flat by passing through the lumen (L). Afterwards, the rear section of the guide tube (11) with spiral structure is inserted into the nose manually or with the help of a holder and thus the number and length of the rings formed in the nose is increased and it is easily caught in the nose with the help of a non-traumatic specific sickle-tip rescue hook (22) and is removed out of the nostril. The drawing which gives the intranasal sagittal sections showing the process steps gradually up to this phase is given in FIG. 9. This sickle tip rescue hook (22) can be manufactured with magnetic feature or can be assembled or disassembled on the end of the magnetic catcher. The hook tip is covered with a soft polymer like silicone and thus it is softened for minimizing the trauma to the mucosa.

Together with the intubation set details of which are given above;

The invention is related to an intubation set which enables treatment of organ damage, stenosis or obstruction such as punctal, canalicular and nasolacrimal duct etc. that provide drainage of liquid in the human body and provides recovery of the liquid drainage ability of these organs in an easy, fast and less invasive manner and enables all surgeons to perform this intervention without requiring experience during long years and ability.

1. An intubation set developed for nasolacrimal drainage for use in treatment of damage, stenosis or obstruction of punctal, canalicular and nasolacrimal duct, characterized by comprising;
a metal probe (13),
a medical tube or medical intubation tube with punctum plug-fixation clamp which has punctum plug-fixation clamp on its end to be used in bicanalicular or monocanalicular intubation processes, has a guide tube (11) on both of its ends or on only one of its end; enables the nasolacrimal passage to be open by remaining in the drainage paths during months,
wherein the guide tube (11) comprising; a guide tube tip (12) that provides inlet from punctums, an opening on it through which the metal probe (13) passes and is directed in the nose and a connection part (B) on the other end where its connection with the medical tube is enabled, wherein the guide tube (11) has a regular or irregular curvature selected from full round or crescent form and wherein there are dimensional lines, markers and colors showing the bending direction of the tube on the guide tube (11), in order to make the surgeon to understand the level of the implanted guide tube (11);
wherein the metal probe (13) enters into the guide tube (11) and is directed in the nose, wherein the metal probe (13) has cylindrical or geometrical shape,
a metal probe holder (14) which prevents orientation of the guide tube (11) to the side and rear directions after it is lowered into the nasal cavity, keeps the upper portion of the guide tube (11) in the same direction, has a groove (15) with adjustable position and form in relation with the structure of the guide tube (11) and enables orientation of the metal probe (13),
a guide clamp (Y) which is located in the middle portion of the guide tube (11), prevents the bottom half of the guide tube (11) from rotating in other directions and shows the surgeon the orientation direction of the guide tube (11) while the guide tube (11) is scrapping towards the nose over the metal probe (13),
a holder to remove the guide tube (11) from the nostril by holding the same.

2. Intubation set according to claim 1, characterized in that; the guide tube (11) is produced from different polymer or monomers with semi-flexible structure and form taking feature, made of poliamid, polyurethane, polyolefin, polysilicates, siloxanes, magnetic materials or derivatives and mixtures thereof.

3. Intubation set according to claim 2, characterized in that; the guide tube (11)
has an external diameter between 0.1-4 mm values,
has lumen (L) internal diameter (L) between 0.1-3.9 mm values,
has a length between 5-30 cm values,
wherein the distance between the guide tube tip (12) and the opening through which the metal probe (13) enters the guide tube lumen (L) is between 3 cm and 30 cm.

4. (canceled)

5. (canceled)

6. Intubation set according to claim 1, characterized in that; the metal probe (13) is manufactured from any material which is flexible, durable, semi-rigid, orientable, non-fragile, smooth reducing friction and with a feature that may give the guide tube (11) its own flat structure and/or from different materials with magnetic feature.

7. (canceled)

8. Intubation set according to claim 1, characterized in that; the metal probe (13)
has a diameter between 0.1-3.9 mm values,
has a length between 3-30 cm values.

9. Intubation set according to claim 1, characterized in that; the metal probe holder (14) has a structure which can be flattened.

10. Intubation set according to claim 1, characterized in that; the guide clamp (Y) is removable.

11. (canceled)

12. Intubation set according to claim 1, characterized in that; the medical tube has a biocompatible structure which can easily take the shape of the channel where it is located and shape the channel.

13. Intubation set according to claim 12, characterized in that;
the medical tube is manufactured from silicone, teflon and/or polyamide.

14. Intubation set according to claim 13, characterized in that; the medical tube
has a diameter between 0.5-1.3 mm values,
has a length between 15-35 cm.

15. Intubation set according to claim 1, characterized in that; the holder is forceps (20), sickle tip rescue hook (22) or material/magnet/electromagnet/holder with magnetic feature (21).

16. Intubation set according to claim 15, characterized in that; the material/magnet/electromagnetic/holder with magnetic feature (21) has an overall diameter between 1 mm and 2 cm values.

17. Intubation set according to claim 15, characterized in that; sickle tip rescue hook (22) has a magnetic feature and the end of the sickle tip rescue hook (22) is covered with a soft polymer.

18. (canceled)

19. Intubation set according to claim 1, characterized in that; the guide tube tip (12),
has a dimension which can be terminated by extending, remaining fixed, narrowing while going to the very end,
has a curved/parabolic (blunt) form, olive-form, mushroom-form or open-ended cylinder form.

20. Intubation set according to claim 19, characterized in that; the guide tube tip (12) is made of material with magnetic feature or without magnetic feature, wherein the material with magnetic feature is a metal which comprises iron, nickel, cobalt, alloys with magnetic feature, neodymium, samarium, and cobalt and/or magnets consisting of rare magnetic metals.

21. Intubation set according to claim 19, characterized in that; the guide tube tip (124) contains magnetic fluid (24) in its lumen (L), a material with magnetic feature in its wall and/or lumen (L).

22. (canceled)

23. (canceled)

24. Intubation set according to claim 21, characterized in that; the guide tube tip (12),
has an external diameter between 0.1-3 mm values,
has an internal lumen between 0.1-2.9 mm values,
wherein end of the guide tube (12) has a length between 0.1 mm and 4 cm values.

25. Intubation set according to claim 21, characterized in that; metal or materials with magnetic feature (23) can remain within the lumen (L) in an injected form and/or in a single form or in segmented form at the guide tube tip (12).

26. (canceled)

27. Intubation set according to claims 1 to 25, characterized in that; it comprises front opening (16a) and rear opening (16b), wherein the distance between the front opening (16a) and the rear opening (16b) is 5-30 cm.

28. (canceled)

29. Intubation set according to claim 1, characterized in that; there is an extended structure (26) in the lumen (L) of the guide tube (11) which is flexible, foldable and can be pushed in the lumen, has continuous and segmented magnetic feature.
